(19) United States
(12) Patent Application Publication
(10) Pub. No.: US 2024/0024735 A1
(43) Pub. Date: Jan. 25, 2024

(54) Treadmill with Running Form Detection Device

(71) Applicant: Michelle Rubio, Bakersfield, CA (US)

(72) Inventor: Michelle Rubio, Bakersfield, CA (US)

(21) Appl. No.: 18/310,657

(22) Filed: May 2, 2023

Related U.S. Application Data
(60) Provisional application No. 63/390,709, filed on Jul. 20, 2022.

(51) Int. Cl. A63B 24/00; A63B 22/02

(52) U.S. Cl. CPC A63B 24/0062; A63B 24/0006; A63B 22/0235; A63B 2220/22; A63B 2024/0071; A63B 2220/803; A63B 2220/802; A63B 2220/40; A63B 2220/58; A63B 2220/56; A63B 2220/836; A63B 2220/62; A63B 2220/52; A63B 2220/53; A63B 2220/36

(57) ABSTRACT

The present invention relates to a novel treadmill with running form detection device. The device is an improved treadmill for detecting a user's gait, landing form, and arm swing, as well as other running biometrics on a treadmill. The treadmill with running form detection device comprises a treadmill having a body component with a plurality of sensors for detecting a user's running form. Typically, the plurality of sensors collect data on the running biometrics of a user, and the output of the sensors is then displayed via a display on the treadmill. Users can identify and correct their running form based on this output.

### CROSS-REFERENCE TO RELATED APPLICATION

The present application claims priority to, and the benefit of, U.S. Provisional Application No. 63/390,709, which was filed on Jul. 20, 2022, and is incorporated herein by reference in its entirety.

### FIELD OF THE INVENTION

The present invention relates generally to the field of treadmills with running form detection devices. More specifically, the present invention relates to an improved treadmill with a plurality of sensors for detecting the gait of a user, the landing form of a user, and the arm swing of the user while running on the treadmill device. Accordingly, this disclosure makes specific reference thereto the present invention. Nonetheless, it is to be appreciated that aspects of the present invention are also equally applicable to other like applications, devices, and methods of manufacture.

### BACKGROUND

By way of background, this invention relates to improvements in treadmills with running form detection devices. Generally, many individuals enjoy running for health and for sport. While running, correct running form is crucial for the prevention of injuries. Accordingly, many running-related injuries occur due to improper gait, landing form, and arm swing. However, these issues are often not identified and/or corrected without observation from a second individual, who may not always be present. This is especially true while on a treadmill.

Gait analysis is the biomechanics systematic study of human motion, using instruments to measure and analyze walk movements. The gait analysis can be used to assess individuals with conditions affecting their ability to walk. Commercial treadmills typically have no gait analysis function. Further, treating therapists typically do not have time to set up a complicated gait recording system, nor can they spend significant money to do so. They also do not need a system that provides an abundance of data, just a few simple parameters that can be compared over time to assess a user's gait and progress.

Therefore, it is an object of this invention to provide a relatively low cost, simple gait-recording, landing form detection, and arm swing detection treadmill device. It is another object to provide a gait measurement, landing form detection, and arm swing detection device that can be easily retrofitted to an existing treadmill.

Therefore, there exists a long-felt need in the art for a treadmill with running form detection device that provides users with an improved treadmill with at least one sensor. There is also a long-felt need in the art for a treadmill with running form detection device that features detection of the user's gait while running on the treadmill. Further, there is a long-felt need in the art for a treadmill with running form detection device that features detection of the user's landing form while running on the treadmill. Moreover, there is a long-felt need in the art for a device that features detection of the user's arm swing while running on the treadmill. Further, there is a long-felt need in the art for a treadmill with running form detection device that detects and displays the user's gait, landing form, and arm swing via a display. Finally, there is a long-felt need in the art for a treadmill with running form detection device that can also detect and display other running biometrics, such as cadence, steps per minute, etc.

The subject matter disclosed and claimed herein, in one embodiment thereof, comprises a treadmill with running form detection device. The device is an improved treadmill for detecting a user's gait, landing form, and arm swing, as well as other running biometrics on a treadmill. The treadmill with running form detection device comprises a treadmill having a body component with a plurality of sensors for detecting a user's running form. Typically, the plurality of sensors collects data on the running biometrics of a user, and the output of the sensors is then displayed via a display on the treadmill. Users can identify and correct their running form based on this output.

In this manner, the treadmill with running form detection device of the present invention accomplishes all of the foregoing objectives and provides users with a device that detects a user's running form while running on a treadmill. The device is a treadmill equipped with a plurality of sensors that collect data and display such data to the user. A user utilizes the device to identify and correct their running form.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a treadmill with running form detection device. The device is an improved treadmill for detecting a user's gait, landing form, and arm swing, as well as other running biometrics on a treadmill. The treadmill with running form detection device comprises a treadmill having a body component with a plurality of sensors for detecting a user's running form. Typically, the plurality of sensors collects data on the running biometrics of a user, and the output of the sensors is then displayed via a display on the treadmill. Users can identify and correct their running form based on this output.

In one embodiment, the treadmill with running form detection device comprises a treadmill having a body component. In the following description, the treadmill device is described in relation to a treadmill. Therefore, the following description omits some prior art components and focuses on the essential components of the treadmill.

In one embodiment, the treadmill comprises a conveyor belt, a supporting plate, a driving mechanism, a control panel with a liquid crystal display, and a plurality of sensors. The conveyor belt is used for a user to walk or run in place. The supporting plate is arranged below the conveyor belt for supporting the conveyor belt. The driving mechanism drives the conveyor belt to run. The plurality of sensors are positioned at different locations on the treadmill device to sense a user's running form, such as a user's gait, landing form, and arm swing, as well as other running biometrics on a treadmill.

In one embodiment, some of the sensors can be fixed with the supporting plate for respectively sensing the user's left and right heel strike. A left and right pulse diagram thus can be obtained from the sensors, and a gait analysis can be performed according to the left and right pulse diagram.

Generally, the sensors on the supporting plate comprise a hall sensor, a magnet, a coupling member, and a fixing member. The sensors couple with the supporting plate via the fixing member. The fixing member may be, but is not limited to, a screw. The hall sensor does not fix with the coupling member but fixes with another mechanism. The magnet fixes with the coupling member. When the user walks or runs, the coupling member will vibrate with the supporting plate, but the hall sensor remains static. The distance between the magnet and the hall sensor is thus varied, and the hall sensor outputs induction voltages depending on the distance between the magnet and the hall sensor so that the left and right pulse diagram can be obtained.

The left and right pulse diagram provides information regarding the relationship between the location of a user's left foot (i.e., the tiptoe or heel portion of the left foot) and the time, and the relationship between the location of a user's right foot (i.e., the tiptoe or heel portion of the right foot) and the time, to determine the gait analysis. In one embodiment, the gait analysis comprises a step number and/or a step frequency of the left and right foot of the user that walks or runs on the conveyor belt. In another embodiment, the gait analysis comprises a step length of the left and right foot of the user that walks or runs on the conveyor belt.

In another embodiment, the treadmill device determines a user's gait analysis via force sensors disposed between the treadmill base and the ground. Specifically, the conveyor belt is connected to the base, typically over at least two rollers within the base. The belt has an upper surface which serves as a walking/running surface for the user. When the user walks/runs on the belt, the load is transferred to the base which in turn, causes each force sensor to emit an electronic signal proportional to the load on the base.

The load of the treadmill plus the user is transferred through the force sensors to the ground. Each force sensor senses the change in the load and transmits a signal proportional to the load to a receiver which in turn, transmits the signal to one or more processing units. The speed of the treadmill belt is measured as either a digital pulse or actual scalar value.

The device then takes the sensor signals and translates them into something that can be interpreted by a computer, in this case, converting the analog signal to digital. The processing performs the gait analysis and hosts the user interface. Communications may be made by wire or wirelessly. Preferably these communications are made wirelessly via USB, Bluetooth, or other wireless communication protocol.

The common gait parameters measured include step length, stride length, symmetry, swing and stance phase percentages. The device measures static load at the center of pressure on each foot at hundreds of times per second, approximating real-time movement measurements. After subtracting the load of the device without the user, the load values and the known geometry of the sensor locations are used to calculate the location and magnitude of the center of pressure for each data point. Then, static analysis principles are used to calculate the gait parameters. One or more processors, along with software installed on the device, performs additional analysis using the force sensor and speed data.

In one embodiment, the data is then displayed via a display of the gait parameters measured by the apparatus, including swing phase time, step length, total weight bearing, base of support, and a map of the center of pressure through the gait cycle, referred to herein as a gait pattern. For example, when only one foot is in contact with the treadmill belt and the other is in swing phase, the lateral movement of the center of pressure is limited to the width of the footprint and is therefore small in velocity. Upon heel strike of the opposing foot, the center of pressure immediately shifts quickly away from the stance foot in one or both directions, depending on the user's gait. Similarly, upon toe off of the first foot, the angular velocity immediately stabilizes to the confines of the opposing foot. Once the location and timing of the heel strike and toe off for each foot are known, a complete analysis of gait parameters can be performed on the data.

Typically, any form of force sensor may be utilized, as is known in the art. Preferably a cantilever beam-style strain gauge is used, which is available commercially. The force sensors are rigidly connected to each other. Typically, four force sensors are employed and arranged in a rectangle, or three force sensors are arranged in a triangle. However, the three-sensor arrangement can have reduced accuracy and precision.

In one embodiment, the plurality of sensors are integrated directly into the base of the treadmill. In this configuration, one end of the force sensor is mounted to the treadmill base and the other end of the force sensor is attached to an adjustable foot that rests on the ground. The adjustable feet are used to level the treadmill, as known in the art.

In another embodiment, modular attachments that house the force sensors are attached to the treadmill base, preferably using mounting points that already exist on the base for the ground support. There are two types of housings: one housing holds a single force sensor, and a second housing holds two force sensors. The single-sensor housings are typically used in the front of the treadmill, one on each side of the mechanism that elevates the head of the treadmill belt to create an inclined walking surface. The dual-sensor housings are typically used across the rear of the treadmill where there's no treadmill structure to straddle.

In one embodiment, the treadmill device comprises a plurality of sensors that measure the critical characteristics of arm swing during walking, running, or other mobile activities. The characteristics may include, but are not limited to, forward arc, backward arc, distance of travel, speed and fluidity. Further, the plurality of sensors can include sensors that automatically record the frequency, amplitude, and treadmill speed of the user's arm swings; by recording and analyzing the frequency and amplitude of the user's many arm swings at different treadmill speeds. Thus, the device provides real-time feedback on the movement of the user's arms while walking or running, using a treadmill.

Relevant physiology and kinematic studies have shown that in order to maintain body balance while running, the magnitude and frequency of the step are roughly proportional to the amplitude and frequency of the arm swing. The faster the arm swing frequency, the faster the frequency of the footsteps. The greater the amplitude of the swing arm, the greater the step size of the step. In addition, when a person turns, the side of the turn is also related to the swing of the arm. When turning to the left, the swing of the right arm is large, and the swing of the left arm is small. Similarly, when turning to the left, the swing of the left arm is large, and the swing of the right arm is small.

Thus, any aspect of arm movement that can be measured can be signaled back to the device user, including, but not limited to, total length of arc, length of forward swing or backward swing, speed of swing, fluidity. Typically, the data is displayed on a display which can be part of the control panel. However, the display can be separate from the control panel, as well. Further, the device may have the capability to compare the left and right arms for measuring consistency of the swing. This may require one or more sensors. It may also have the capability to store arm swing data for downloading and later comparison. It may also have the capability to signal a remote observer, if desired. In one embodiment, the device may have remote signaling or wireless capability for immediate feedback in real-time to the user.

In one embodiment, the device comprises sensors for sensing the motion of an arm of a user during a movement activity. Specifically, the motion sensing means or the one or more sensors may comprise one or more accelerometers and/or ultrasonic motion sensors configured to measure at least one of: a speed of the arm of the user, a length of a swing arc of the arm of the user, a forward swing of the arm of the user, a backward swing of the arm of the user, and a fluidity of movement of the arm of the user.

In one embodiment, the sensors to track a user's arm swing are located on the treadmill device, positioned on either side of the upper arms of the treadmill and/or at the front of the treadmill. These stationary sensors would comprise ultrasonic motion sensors to measure arm swing of a user. However, arm swing does not have to be measured via stationary sensors located on the treadmill but can also be measured via sensors positioned on the user.

For example, in one embodiment, the sensors to track a user's arm swing comprise a strap or band which attaches to the wrist of the user. The strap or band carries a control unit comprising a multi-color status light emitting diode (LED) adapted to provide the user with visual feedback on the device's internal processes, such as battery life status, program run mode, etc.; a universal serial bus (“USB”) port that functions as a power supply and data communication route when connected to a computer; a toggle switch that enables the user to specify auditory feedback, in the form of a continuous tone, or tactile feedback in the form of localized vibration around the wrist; and a power switch. In this embodiment, the strap or band can be formed from any suitable stretchable fabric, such as neoprene, spandex, and elastane. Alternatively, in one or more other embodiments, the band could be formed from a generally non-stretchable fabric and can be provided with latching means or clasp means for allowing the band to be split into two portions.

In another embodiment, the sensors can be in the form of a handheld device that is simply held by the user in his or her hand. In this embodiment, the strap or band is omitted entirely, and the user merely holds the sensor unit in his or her hand. In the embodiments wherein the sensors are positioned on the user while running/walking on a treadmill, the sensors would comprise accelerometers to measure arm swing of a user.

In one embodiment, the sensor for detecting gait, landing form, and arm swing comprise a central control unit, motion sensing means that are operatively coupled to the central control unit, an audio signaling device, and a tactile signaling device that are operatively coupled to the central control unit, device memory operatively coupled to the central control unit for storing data during the processing thereof, and a power supply. The primary power supply or power source for the sensor may be in the form of a battery. The sensor may also comprise a wired power input for charging the battery inside the unit, or for providing supplementary power to the unit when the battery has a low charge. The device memory may comprise volatile memory, non-volatile memory, or a combination thereof.

In yet another embodiment, the treadmill with running form detection device comprises a plurality of indicia.

It should be noted that the treadmill device is not limited to the arm swing detecting sensors, the gait sensors, and the landing form sensors described above. Any biomedical instrument that can be used to monitor the user's physical indicators during exercise can be used in this treadmill device.

In yet another embodiment, a method of detecting a user's running form while running on a treadmill is disclosed. The method includes the steps of providing a treadmill with running form detection device comprising a treadmill body component with a plurality of sensors. The method also comprises stepping onto the treadmill tread. Further, the method comprises starting the treadmill and activating the plurality of sensors. Additionally, the method comprises running on the treadmill device. The method comprises collecting running form data from the plurality of sensors. The method also comprises displaying the collected data from the plurality of sensors via a display on the treadmill. Finally, the method comprises analyzing the displayed data and identifying and correcting a user's running form.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one embodiment of the treadmill with running form detection device of the present invention in use in accordance with the disclosed architecture;

FIG. 2A illustrates a perspective view of one embodiment of the treadmill with running form detection device of the present invention showing the hall sensor for measuring a user's gait in accordance with the disclosed architecture;

FIG. 2B illustrates a perspective view of one embodiment of the treadmill with running form detection device of the present invention showing the force sensors for measuring a user's gait in accordance with the disclosed architecture;

FIG. 2C illustrates a perspective view of one embodiment of the treadmill with running form detection device of the present invention showing the arm sensor for measuring a user's arm swing as a handheld device in accordance with the disclosed architecture;

FIG. 2D illustrates a perspective view of one embodiment of the treadmill with running form detection device of the present invention showing the arm sensor on a wrist band in accordance with the disclosed architecture;

FIG. 2E illustrates a perspective view of one embodiment of the treadmill with running form detection device of the present invention showing a sensor for measuring a user's gait, landing form and arm swing in accordance with the disclosed architecture; and

FIG. 3 illustrates a flowchart showing the method of detecting a user's running form while running on a treadmill in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a treadmill with running form detection device that provides users with an improved treadmill with at least one sensor. There is also a long-felt need in the art for a treadmill with running form detection device that features detection of the user's gait while running on the treadmill. Further, there is a long-felt need in the art for a treadmill with running form detection device that features detection of the user's landing form while running on the treadmill. Moreover, there is a long-felt need in the art for a device that features detection of the user's arm swing while running on the treadmill. Further, there is a long-felt need in the art for a treadmill with running form detection device that detects and displays the user's gait, landing form, and arm swing via a display. Finally, there is a long-felt need in the art for a treadmill with running form detection device that can also detect and display other running biometrics, such as cadence, steps per minute, etc.

The present invention, in one exemplary embodiment, is a novel treadmill with running form detection device. The device is an improved treadmill for detecting a user's gait, landing form, and arm swing, as well as other running biometrics on a treadmill. The treadmill with running form detection device comprises a treadmill having a body component with a plurality of sensors for detecting a user's running form. Typically, the plurality of sensors collect data on the running biometrics of a user, and the output of the sensors is then displayed via a display on the treadmill. Users can identify and correct their running form based on this output. The present invention also includes a novel method of detecting a user's running form while running on a treadmill. The method includes the steps of providing a treadmill with running form detection device comprising a treadmill body component with a plurality of sensors. The method also comprises stepping onto the treadmill tread. Further, the method comprises starting the treadmill and activating the plurality of sensors. Additionally, the method comprises running on the treadmill device. The method comprises collecting running form data from the plurality of sensors. The method also comprises displaying the collected data from the plurality of sensors via a display on the treadmill. Finally, the method comprises analyzing the displayed data and identifying and correcting a user's running form.

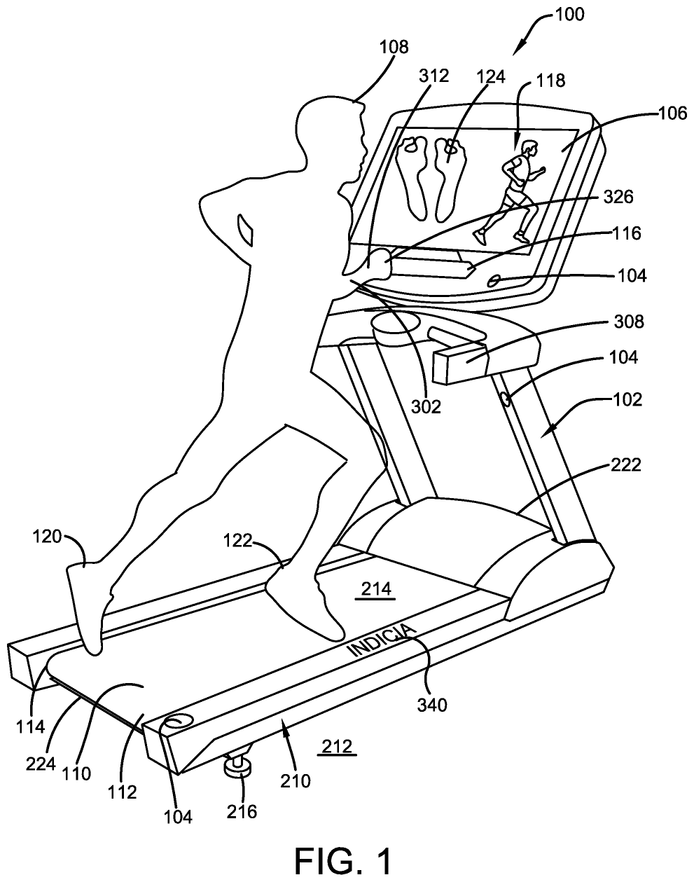

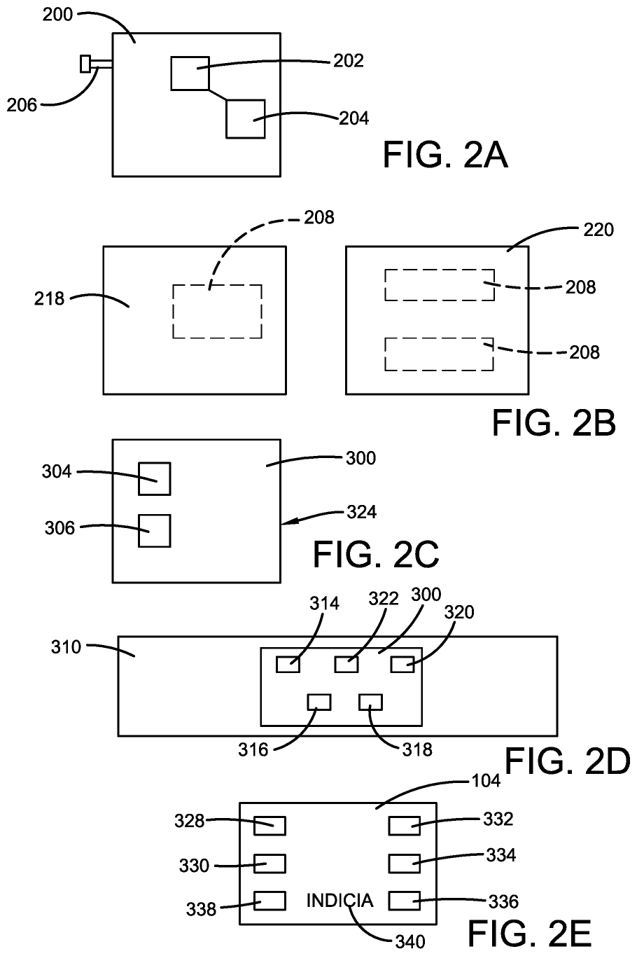

Referring initially to the drawings, FIGS. 1 and 2A to 2E illustrate a perspective view of one embodiment of the treadmill with running form detection device 100 of the present invention. In the present embodiment, the treadmill with running form detection device 100 is an improved treadmill with running form detection device 100 that detects a user's 108 running form while running on a treadmill 102. Specifically, the treadmill with running form detection device 100 comprises a body component 102 with a plurality of sensors 104 to detect a user's gait, landing form, and arm swing, as well as other running biometrics on a treadmill 102. Typically, the plurality of sensors 104 collect data on the running biometrics of a user 108, and the output of the sensors 104 is then displayed via a display 106 on the treadmill 102. Users 108 can identify and correct their running form based on this output.

Generally, the treadmill with running form detection device 100 comprises a treadmill having a body component 102. In the following description, the treadmill device 100 is described in relation to a treadmill 102. Therefore, the following description omits some prior art components and focuses on the essential components of the treadmill 102.

Typically, a treadmill 102 comprises a conveyor belt 110, a supporting plate 112, a driving mechanism 114, a control panel 116 with a liquid crystal display 118, and a plurality of sensors 104. The conveyor belt 110 is used for a user to walk or run in place. The supporting plate 112 is arranged below the conveyor belt 110 for supporting the conveyor belt 110. The driving mechanism 114 drives the conveyor belt 110 to move. The plurality of sensors 104 are positioned at different locations on the treadmill device 100 to sense a user's running form, such as a user's gait, landing form, and arm swing, as well as other running biometrics on a treadmill 102.

In one embodiment, the plurality of sensors 104 are utilized to detect a user's gait and/or landing form. These sensors 104 can be motion sensors, ultrasonic sensors, accelerometers, etc., or any other suitable sensing components that can sense a user's gait and/or landing form while running/walking on a treadmill 102. These sensors 104 can be located at different positions on and around the treadmill 102, depending on the biometric they intend to detect and measure. For example, when detecting a user's gait and/or landing form, some of the sensors 104 can be fixed with the supporting plate 112, for respectively sensing the user's left 120 and right 122 heel strike. A left and right pulse diagram 124 thus can be obtained from the sensors 104, and a gait analysis can be performed according to the left and right pulse diagram 124.

As shown in FIG. 2A, the sensors 104 on the supporting plate 112 comprise a hall sensor 200, a magnet 202, a coupling member 204, and a fixing member 206. The sensors 104 couple with the supporting plate 112 via the fixing member 206. The fixing member 206 may be, but is not limited to, a screw or any other suitable fixing member as is known in the art. The hall sensor 200 does not fix with the coupling member 204 but fixes with another mechanism. The magnet 202 fixes with the coupling member 204. When the user 108 walks or runs, the coupling member 204 will vibrate with the supporting plate 112 but the hall sensor 200 remains static. The distance between the magnet 202 and the hall sensor 200 is thus varied, and the hall sensor 200 outputs induction voltages depending on the distance between the magnet 202 and the hall sensor 200, so that the left and right pulse diagram 124 can be obtained.

The left and right pulse diagram 124 provides information regarding the relationship between the location of a user's left foot 120 (i.e., the tiptoe or heel portion of the left foot 120) and the time, and the relationship between the location of a user's right foot 122 (i.e., the tiptoe or heel portion of the right foot 122) and the time, to determine the gait analysis. In one embodiment, the gait analysis comprises a step number and/or a step frequency of the left 120 and right 122 foot of the user 108 that walks or runs on the conveyor belt 110. In another embodiment, the gait analysis comprises a step length of the left 120 and right 122 foot of the user 108 that walks or runs on the conveyor belt 110.

In another embodiment shown in FIG. 2B, the treadmill device 100 determines a user's gait analysis via force sensors 208 disposed between the treadmill base 210 and the ground 212. Specifically, the conveyor belt 110 is connected to the base 210, typically over at least two rollers within the base 210. The belt 110 has an upper surface 214 which serves as a walking/running surface for the user 108. When the user 108 walks/runs on the belt 110 the load is transferred to the base 210, which in turn causes each force sensor 208 to emit an electronic signal proportional to the load on the base 210.

The load of the treadmill 102 plus the user 108 is transferred through the force sensors 208 to the ground 212. Each force sensor 208 senses the change in the load and transmits a signal proportional to the load to a receiver which in turn transmits the signal to one or more processing units. The speed of the treadmill belt 110 is then measured as either a digital pulse or actual scalar value.

The device 100 then takes the sensor signals and translates them into something that can be interpreted by a computer, in this case, converting the analog signal to digital. The processing performs the gait analysis and/or landing form analysis and hosts the user interface. Communications may be made by wire or wirelessly. Preferably these communications are made wirelessly via USB, Bluetooth, or other wireless communication protocol.

Accordingly, the common gait parameters measured include step length, stride length, symmetry, swing, and stance phase percentages. The device 100 measures static load at the center of pressure on each foot at hundreds of times per second, approximating real-time movement measurements. After subtracting the load of the device 100 without the user 108, the load values and the known geometry of the sensor 208 locations are used to calculate the location and magnitude of the center of pressure for each data point. Then, static analysis principles are used to calculate the gait parameters. One or more processors, along with software installed on the device 100 performs additional analysis using the force sensor 208 and speed data.

Furthermore, the data is then displayed via a display 106 of the gait parameters measured by the apparatus 100, including swing phase time, step length, total weight bearing, base of support, and a map of the center of pressure through the gait cycle, referred to herein as a gait pattern. For example, when only one foot is in contact with the treadmill belt 110 and the other is in swing phase, the lateral movement of the center of pressure is limited to the width of the footprint and is therefore, small in velocity. Upon heel strike of the opposing foot, the center of pressure immediately shifts quickly away from the stance foot in one or both directions, depending on the user's gait. Similarly, upon toe off of the first foot, the angular velocity immediately stabilizes to the confines of the opposing foot. Once the location and timing of the heel strike and toe off for each foot are known, a complete analysis of gait parameters can be performed on the data.

Typically, any form of force sensor 208 may be utilized, as is known in the art. Preferably a cantilever beam-style strain gauge is used, which is available commercially. The force sensors 208 are rigidly connected to each other. Typically, four force sensors 208 are employed, arranged in a rectangle, or three force sensors 208 arranged in a triangle. However, the three-sensor arrangement can have reduced accuracy and precision when compared to the four force sensors 208.

In another embodiment, the plurality of sensors 208 are integrated directly into the base 210 of the treadmill 102. In this configuration one end of the force sensor 208 is mounted to the treadmill base 210 and the other end of the force sensor 208 is attached to an adjustable foot 216 that rests on the ground 212. The adjustable feet 216 are used to level the treadmill 102, as known in the art.

In yet another embodiment, modular attachments that house 218 and 220 the force sensors 208 are attached to the treadmill base 210, preferably using mounting points that already exist on the base 210 for the ground support. There are two types of housings 218 and 220: one housing 218 holds a single force sensor 208, and a second housing 220 holds two force sensors 208. The single-sensor housings 218 are typically used in the front 222 of the treadmill 102, one on each side of the mechanism that elevates the head of the treadmill belt 110 to create an inclined walking surface. The dual-sensor housings 220 are typically used across the rear 224 of the treadmill 102 where there is no treadmill structure to straddle.

Additionally, the treadmill device 100 comprises a plurality of sensors 104 that measure the critical characteristics of arm swing during walking, running, or other mobile activities. The characteristics may include, but are not limited to, forward arc, backward arc, distance of travel, speed, and fluidity. Further, the plurality of sensors 104 can include sensors 300 that automatically record the frequency, amplitude, and treadmill speed of the user's arm swing 302; by recording and analyzing the frequency and amplitude of the user's many arm swings 302 at different treadmill speeds. Thus, the device 100 provides real-time feedback on the movement of the user's arms 302 while walking or running using a treadmill 102.

Relevant physiology and kinematic studies have shown that in order to maintain body balance while running, the magnitude and frequency of the step are roughly proportional to the amplitude and frequency of the arm swing. The faster the arm swing frequency, the faster the frequency of the footsteps. The greater the amplitude of the arm swing, the greater the step size of the step. In addition, when a person turns, the side of the turn is also related to the swing of the arm. When turning to the left, the swing of the right arm is large and the swing of the left arm is small. When turning to the right, the swing of the left arm is large, and the swing of the right arm is small.

Thus, any aspect of arm movement that can be measured can be signaled back to the device user 108, including but not limited to total length of arc, length of forward swing or backward swing, speed of swing, fluidity. Typically, the data is displayed on a display 106 which can be part of the control panel 116. However, the display 106 can be separate from the control panel 116, as well. Further, the device 100 may have the capability to compare the left and right arms 302 of a user 108 for measuring consistency of swing. This may require one or more sensors 300. It may also have the capability to store arm swing data for downloading and later comparison. It may also have the capability to signal a remote observer, if desired. In one embodiment, the device 100 may have remote signaling or wireless capability for immediate feedback in real-time to the user 108.

In one embodiment as shown in FIGS. 2C-D, the device 100 comprises sensors 300 for sensing the motion of an arm 302 of a user 108 during a movement activity. Specifically, the motion sensing means or the one or more sensors 300 may comprise one or more accelerometers 304 and/or ultrasonic motion sensors 306 configured to measure at least one of: a speed of the arm 302 of the user 108, a length of a swing arc of the arm 302 of the user 108, a forward swing of the arm 302 of the user 108, a backward swing of the arm 302 of the user 108, and a fluidity of movement of the arm 302 of the user 108.

In one embodiment, the sensors 300 to track a user's arm swing are located on the treadmill device 100, positioned on either side of the upper arms 308 of the treadmill 102 and/or at the front 222 of the treadmill 102. These stationary sensors 300 would comprise ultrasonic motion sensors 306 to measure arm swing of a user 108. However, arm swing doesn't have to be measured via stationary sensors 300 located on the treadmill 102 but can also be measured via sensors positioned on the user 108.

For example, in one embodiment, the sensors 300 to track a user's arm swing comprise a strap or band 310 which attaches to the wrist 312 of the user 108, shown in FIG. 2D. The strap or band 310 carries a control unit 314 comprising a multi-color status light emitting diode (LED) 316 adapted to provide the user 108 with visual feedback on the device's internal processes, such as battery life status, program run mode, etc.; a universal serial bus (“USB”) port 318 that functions as a power supply and data communication route when connected to a computer; a toggle switch 320 that enables the user 108 to specify auditory feedback, in the form of a continuous tone, or tactile feedback, in the form of localized vibration around the wrist; and a power switch 322. In this embodiment, the strap or band 310 can be formed from any suitable stretchable fabric, such as neoprene, spandex, and elastane. Alternatively, in one or more other embodiments, the band 310 could be formed from a generally non-stretchable fabric and be provided with latching means or clasp means for allowing the band 310 to be split into two portions.

In another embodiment, the sensors 300 can be in the form of a handheld device 324 that is simply held by the user 108 in his or her hand 326, shown in FIG. 2C. In this embodiment, the strap or band 310 is omitted entirely and the user 108 merely holds the sensor unit 324 in his or her hand 326. In the embodiments wherein the sensors 300 are positioned on the user 108 while running/walking on a treadmill 102, the sensors 300 would comprise accelerometers 304 to measure arm swing of a user 108.

In one embodiment shown in FIG. 2E, the sensors 104 for detecting gait, landing form, and arm swing comprise a central control unit 328, motion sensing means 330 that are operatively coupled to the central control unit 328, an audio signaling device 332 and a tactile signaling device 334 that are operatively coupled to the central control unit 328, device memory 336 operatively coupled to the central control unit 328 for storing data during the processing thereof, and a power supply 338. The primary power supply or power source 338 for the sensors 104 may be in the form of a battery (not shown). The sensors 104 may also comprise a wired power input for charging the battery inside the unit, or for providing supplementary power to the unit when the battery has a low charge. The device memory 336 may comprise volatile memory, non-volatile memory, or a combination thereof.

In yet another embodiment, the treadmill with running form detection device 100 comprises a plurality of indicia 340. The body component 102 of the device 100 may include advertising, trademark, other letters, designs, or characters, printed, painted, stamped, or integrated into the body component 102, or any other indicia 340 as is known in the art. Specifically, any suitable indicia 200 as is known in the art can be included, such as, but not limited to, patterns, logos, emblems, images, symbols, designs, letters, words, characters, animals, advertisements, brands, etc., that may or may not be running, treadmill, or brand related.

It should be noted that the treadmill device 100 is not limited to the arm swing detecting sensors 104, the gait sensors 104, and the landing form sensors 104 described above. Any biomedical instrument that can be used to monitor the user's physical indicators during exercise can be used in this treadmill device 100.

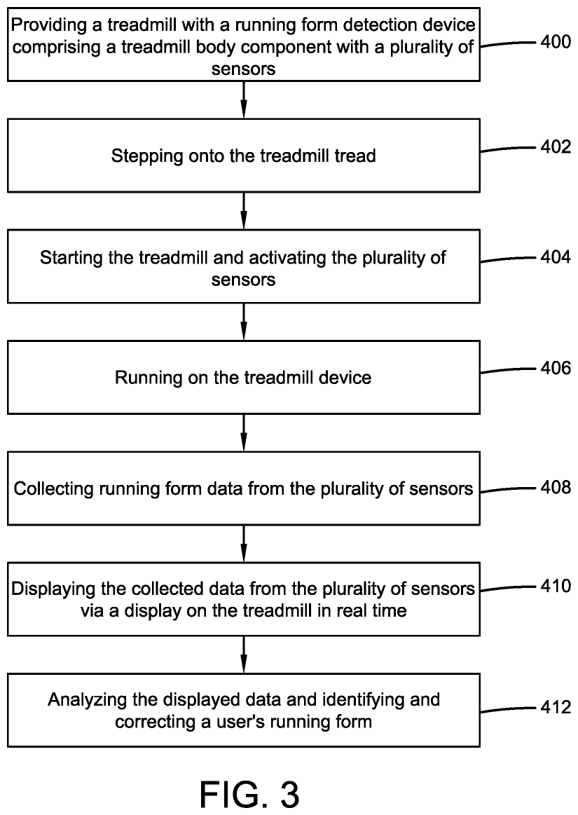

FIG. 3 illustrates a flowchart of the method of detecting a user's running form while running on a treadmill. The method includes the steps of at 400, providing a treadmill with running form detection device comprising a treadmill body component with a plurality of sensors. The method also comprises at 402, stepping onto the treadmill tread. Further, the method comprises at 404, starting the treadmill and activating the plurality of sensors. Additionally, the method comprises at 406, running on the treadmill device. The method comprises at 408, collecting running form data from the plurality of sensors. The method also comprises at 410, displaying the collected data from the plurality of sensors via a display on the treadmill in real-time. Finally, the method comprises at 412, analyzing the displayed data and identifying and correcting a user's running form.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “treadmill with running form detection device”, “treadmill device”, and “device” are interchangeable and refer to the treadmill with running form detection device 100 of the present invention.

Notwithstanding the foregoing, the treadmill with running form detection device 100 of the present invention can be of any suitable size and configuration as is known in the art without affecting the overall concept of the invention, provided that it accomplishes the above-stated objectives. One of ordinary skill in the art will appreciate that the treadmill with running form detection device 100 as shown in FIGS. 1-3 are for illustrative purposes only, and that many other sizes and shapes of the treadmill with running form detection device 100 are well within the scope of the present disclosure. Although the dimensions of the treadmill with running form detection device 100 are important design parameters for user convenience, the treadmill with running form detection device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A treadmill with running form detection device that detects a user's running form while running or walking on a treadmill, the treadmill with running form detection device comprising:
a body component; and
a plurality of sensors;
wherein the plurality of sensors are positioned on the body component; and
further wherein the plurality of sensors detect a user's gait, landing form and arm swing while a user is running or walking on the body component.

2. The treadmill with running form detection device of claim 1, wherein the plurality of sensors collect data on the user's gait, landing form and arm swing, and output of the plurality of sensors is then displayed via a display on the body component.

3. The treadmill with running form detection device of claim 2, wherein the body component comprises a conveyor belt for a user to walk or run in place, a supporting plate arranged below the conveyor belt for support, a driving mechanism that drives the conveyor belt, and a control panel with a liquid crystal display.

4. The treadmill with running form detection device of claim 3, wherein the plurality of sensors comprise motion sensors, ultrasonic sensors, or accelerometers.

5. The treadmill with running form detection device of claim 4, wherein some of the plurality of sensors are fixed with the supporting plate for sensing a user's left and right heel strike to create a left and right pulse diagram.

6. The treadmill with running form detection device of claim 5, wherein the plurality of sensors on the supporting plate each comprise a hall sensor, a magnet, a coupling member, and a fixing member, wherein each of the plurality of sensors couple with the supporting plate via the fixing member, and the hall sensor does not fix with the coupling member but the magnet fixes with the coupling member, such that when the user walks or runs, the coupling member will vibrate with the supporting plate but the hall sensor remains static.

7. The treadmill with running form detection device of claim 6, wherein some of the plurality of sensors comprises force sensors disposed between a treadmill base and a ground, wherein a load of the body component plus the user is transferred through the force sensors to the ground and the force sensors sense change in the load and transmits a signal proportional to the load to a receiver which in turn transmits the signal to a processing unit to determine a user's gait or landing form.

8. The treadmill with running form detection device of claim 7, wherein common gait parameters measured include step length, stride length, symmetry, swing and stance phase percentages.

9. The treadmill with running form detection device of claim 8, wherein data displayed on the display for gait or landing form includes swing phase time, step length, total weight bearing, base of support, and a map of a center of pressure through a gait cycle.

10. The treadmill with running form detection device of claim 9, wherein some of the plurality of sensors measure critical characteristics of arm swing during walking or running on a treadmill.

11. The treadmill with running form detection device of claim 10, wherein characteristics of arm swing include forward arc, backward arc, distance of travel, speed and fluidity.

12. The treadmill with running form detection device of claim 11, wherein the some of the plurality of sensors which measure arm swing are positioned on either side of upper arms of the treadmill and at front of the treadmill.

13. The treadmill with running form detection device of claim 11, wherein the some of the plurality of sensors which measure arm swing comprise a strap which attaches to a user's wrist, wherein the strap comprises a control unit with LED for visual feedback, a USB port for power and power switch.

14. The treadmill with running form detection device of claim 11, wherein the some of the plurality of sensors which measure arm swing comprise a handheld device that comprises an accelerometer.

15. The treadmill with running form detection device of claim 11, wherein the plurality of sensors comprise a central control unit, motion sensing means that are operatively coupled to the central control unit, an audio signaling device and a tactile signaling device that are operatively coupled to the central control unit, a device memory operatively coupled to the central control unit for storing data during processing, and a power supply.

16. A treadmill with running form detection device that detects a user's running form while running or walking on a treadmill, the treadmill with running form detection device comprising:
a body component comprising a conveyor belt for a user to walk or run in place;
a supporting plate arranged below the conveyor belt for support;
a driving mechanism that drives the conveyor belt;
a control panel with a liquid crystal display; and
a plurality of sensors comprising motion sensors, ultrasonic sensors, or accelerometers;
wherein the plurality of sensors are positioned on the body component;
wherein the plurality of sensors detect a user's gait, landing form and arm swing while a user is running or walking on the body component;
wherein some of the plurality of sensors are fixed with the supporting plate for sensing a user's left and right heel strike to create a left and right pulse diagram;
wherein some of the plurality of sensors comprises force sensors disposed between a treadmill base and a ground, to determine a user's gait or landing form;
wherein some of the plurality of sensors measure critical characteristics of arm swing during walking or running on a treadmill;
wherein the plurality of sensors collect data on the user's gait, landing form and arm swing, and output of the plurality of sensors is then displayed via a display on the body component; and
further wherein the display provides real-time feedback on movement of a user's arms and a user's gait and landing form, while walking or running on a treadmill.

17. The treadmill with running form detection device of claim 16 further comprising a plurality of indicia.

18. The treadmill with running form detection device of claim 16, wherein the some of the plurality of sensors which measure arm swing are positioned on either side of upper arms of the treadmill and at front of the treadmill.

19. The treadmill with running form detection device of claim 16, wherein the some of the plurality of sensors which measure arm swing comprise a strap which attaches to a user's wrist or a handheld device that comprises an accelerometer.

20. A method of detecting a user's running form while running on a treadmill, the method comprising the following steps:
providing a treadmill with running form detection device comprising a treadmill body component with a plurality of sensors;
stepping onto the treadmill tread;
starting the treadmill and activating the plurality of sensors;
running on the treadmill device;
collecting running form data from the plurality of sensors;
displaying the collected data from the plurality of sensors via a display on the treadmill in real-time; and
analyzing the displayed data and identifying and correcting a user's running form.
